(19) United States
(12) Patent Application Publication
(10) Pub. No.: US 2006/0271023 A1
(43) Pub. Date: Nov. 30, 2006

(54) Aesthetic treatment installation using a rationalised layout

(76) Inventors: Jean-Daniel Mondin, Levallois-Perret (FR); Jean-Francois Mondin, Levallois-Perret (FR)

Correspondence Address: BACHMAN & LAPOINTE, P.C., 900 CHAPEL STREET, SUITE 1201, NEW HAVEN, CT, 06510, US

(21) Appl. No.: 11/431,335

(22) Filed: May 9, 2006

(30) Foreign Application Priority Data
May 13, 2005 (FR) 05 04873

(51) Int. Cl. A61B 17/00

(52) U.S. Cl. 606/1

(57) ABSTRACT

An aesthetic treatment installation includes a treatment table, treatment appliances and accessories. The installation of the invention includes storage spaces associated with the treatment table, some of which are dedicated to the appliances and others to their accessories, with a control panel placed at the head of the treatment table containing the control devices of the appliances, and sockets for connection of the accessories, together with connections located under the treatment table for connection of the control devices to the corresponding inputs of the appliances and of the sockets to the corresponding outputs of these appliances.

### BACKGROUND OF THE INVENTION

(1) Field of the Invention

In general, the invention concerns equipment (5) used for aesthetic treatments.

More precisely, the invention concerns an aesthetic treatment installation that includes a treatment table with at least one head and a median part, with treatment appliances, such as stimulation or ionisation appliances connected to an electrical source, accessories such as electrodes including power leads and equipping the respective treatment appliances, and with control and/or adjustment devices for the treatment appliances, the latter being equipped with inputs and outputs for control and/or adjustment signals, presenting output signals for delivery to their respective accessories.

(2) Prior Art

In general, these treatment cabins are relatively cramped.

The appliances used in a treatment cabin are traditionally equipped with individual supports, either fixed or on rollers, positioned between the treatment table and one wall of the cabin, and connected to this wall for their electrical supply.

Given the space taken up by each appliance, a treatment cabin is able to accommodate only a very limited number of appliances before it becomes excessively cluttered, thus limiting the movements of the practitioner and sometimes causing inconvenience for the clients.

Moreover, the practitioner is always obliged to turn around in order to operate the various control and/or adjustment buttons of the appliances, and to reach the accessories with which these appliances are equipped.

All of these conditions combine to mean that the operation of treatment cabins can represent considerable inconvenience for both the practitioner and the client.

### SUMMARY OF THE INVENTION

In this context, this present invention has as its objective to improve the ergonomic design of these aesthetic treatment installations.

To this end, the installation of the invention, which is also in accordance with the generic description provided in the above preamble, is essentially characterised in that it also includes first treatment-table resources associated with the treatment table, each of which accommodates at least one treatment appliance, with second storage resources provided at the head of the treatment table and designed to accommodate the accessories, and a control panel placed at the head of the treatment table and on which are grouped the control and/or adjustment devices, this panel being also equipped with sockets for connection of the power leads, and where this installation also includes connection resources positioned under the treatment table to connect the control and/or adjustment devices of the panel to the corresponding inputs of the appliances, and the sockets of the panel to the corresponding outputs of these appliances.

The first storage resources, which can be composed of boxes or drawers, are mounted, for example, under a lower face of the median part of the treatment table.

The second storage resources, which can be composed of drawers or small tables for example, can be mounted under a lower face of the head of the treatment table, for example.

In addition, the control panel can include display devices capable of displaying the operational state of the various appliances, with these display devices being connected to the respective appliances by the connection resources.

The treatment installation of the invention can also include a display screen placed at the head of the treatment table and built into the control panel, for example.

This display screen can be composed of a touch screen and be connected to a centralised computer system equipping the installation.

### BRIEF DESCRIPTION OF THE DRAWINGS

Other characteristics and advantages of the invention will emerge more clearly from the description that follows, which is provided for guidance only and in no way limiting, with reference to the appended drawings, in which:

FIG. 1 is a partial side view in perspective of an installation according to the invention,

FIG. 2 is a partial exploded view of such an installation,

FIG. 3A is a rear view in perspective of such an installation,

FIG. 3B is a rear view in perspective of a first variant of such an installation; and

FIG. 3C is a rear view in perspective of a second variant of such an installation.

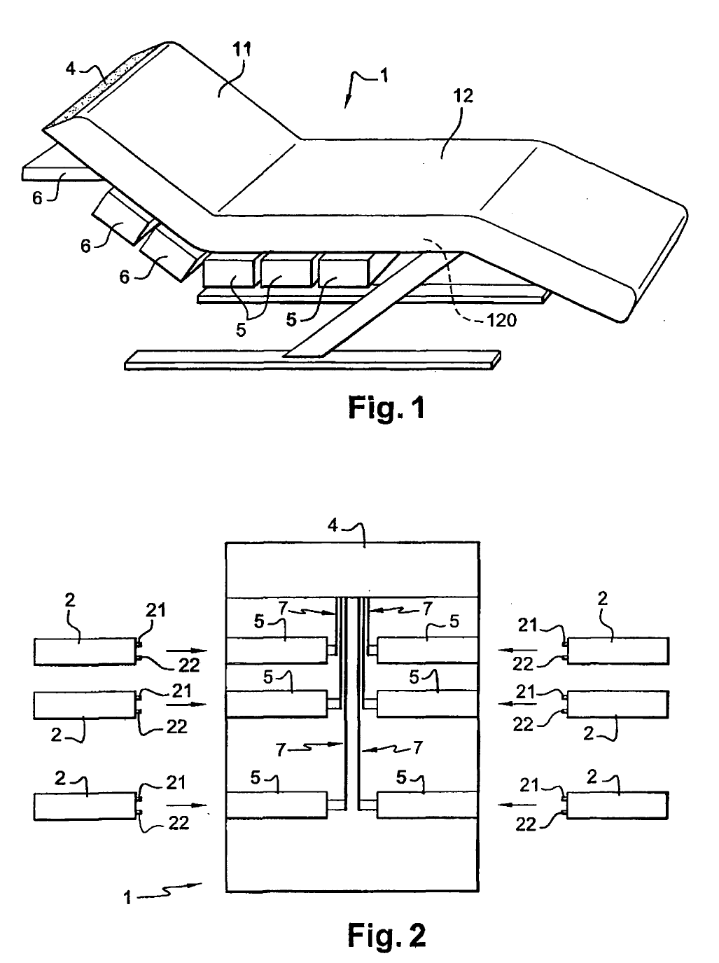

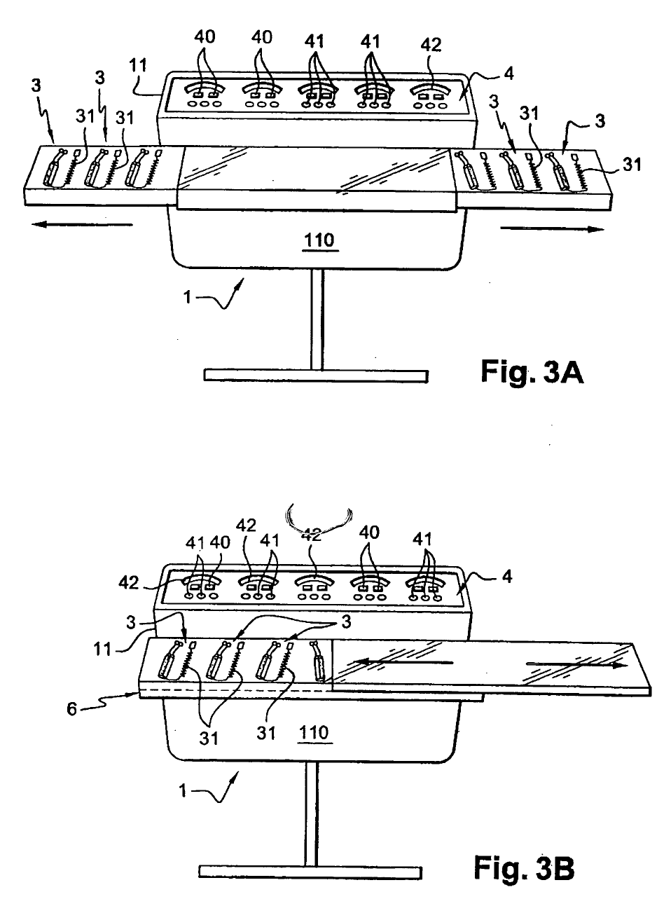

### DETAILED DESCRIPTION OF THE PREFERRED EMBODIMENT(S)

As stated previously, the invention concerns an aesthetic treatment installation that includes a treatment table 1, treatment appliances 2, accessories 3 equipping the respective treatment appliances, and devices 40 for the control and/or adjustment of these treatment appliances 2.

The treatment appliances 2, which can, for example, be electrical stimulation devices, ionisation appliances or indeed epilation appliances, are connected to an electrical source, in general to the mains supply, by means of individual mains cables.

The accessories 3 which equip the various appliances 2 are, for example, composed of heating electrodes or elements, and include power leads 31 used to connect them to the appliances to which they are fitted.

In general, each of these treatment appliances 2 is equipped, firstly, with one or more inputs 21 designed to receive control and/or adjustment signals coming from one or more control and/or adjustment devices 40, and secondly with one or more outputs 22 at which this appliance presents output signals for delivery to the accessory or accessories 3 fitted to them.

The installation of the invention also includes storage spaces 5 associated with the treatment table 1 and dedicated to the treatment appliances 2, storage spaces 6 associated with the treatment table 1 and dedicated to the accessories 3, a control panel 4, and connections 7 between the control panel 4 and the various appliances 2.

The storage spaces 5, each of which is designed to receive one or more appliances 2, are composed, for example, of boxes or drawers mounted under the lower face 120 of the median part 12 of the treatment table 1.

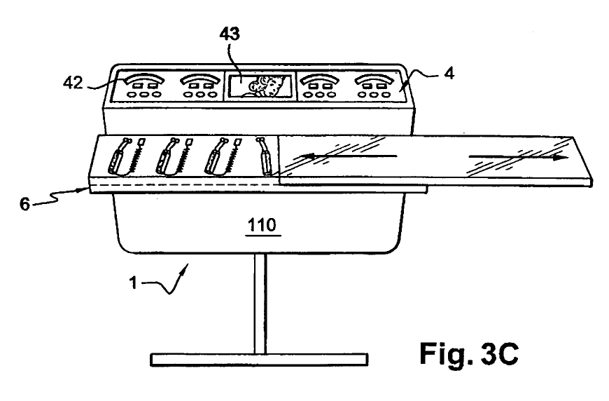

The storage spaces 6, each of which is typically designed to receive several accessories 3, are composed of drawers (FIG. 3A) or of small tables (FIG. 3B) for example, mounted under the lower face 110 of the head 11 of the treatment table 1.

The control panel 4 is placed at the head 11 of the treatment table 1, such as on the upper section of the bed-head 11 for example, and brings together the control and/or adjustment devices 40 of the various appliances 2.

This panel 4 is also equipped with sockets 41 to allow connection of the power leads 31, and preferably includes display devices 42 capable of displaying the operational state of the various appliances 2.

The various connections 7, such as those composed of cables of possibly diverse natures for example, are located under the treatment table 1 and secured, where appropriate, to the lower faces 110, 120 of the treatment table.

These connections are used to connect the Control and/or adjustment devices 40 of the treatment table 4 to the corresponding inputs 21 of the appliances, to connect the sockets 41 of the panel 4 to the corresponding outputs 22 of these appliances 2, and to connect the display devices 42 to specialised outputs of these appliances.

The installation of the invention can be equipped with a single multiple electrical mains socket located under the treatment table 1, to allow the simultaneous electrical connection of all the appliances 2, and connected to the mains supply by a single power cable.

Finally, as shown in FIG. 3C, this installation can also be equipped with a display screen 43 placed at the head 11 of the treatment table 1 and built into the control panel 4 for example.

This screen can be composed of a flat screen of the LCD type for example, approximately square and measuring about 10 cm on each side.

The practitioner, being seated at the rear of the treatment table 1 thus faces the screen 43 and can therefore easily receive information presented on this screen.

As an example, the screen 43 can supply information on the client installed on the treatment table 1, including name and address, consumption and living habits, the history of treatment previously administered in the same beauty salon, the history of the products already purchased, a diagnosis of the skin condition, a diagnosis concerning body treatments and/or a record of medical counter-indications (allergies, etc.).

The screen can also show, in video, the treatment sequences to be administered by the practitioner. This process can thus allows the administration of the various treatments in the salons to be made more reliable and harmonious, and training courses followed by each practitioner to be completed effectively. To this end, all of the administration of each treatment with the use of the products can be presented on the screen in real time to the practitioner, who then only needs to follow this visual information as closely as possible.

The screen can also be used to provide the practitioner with information that is controlled by a centralised computer system equipping the installation, and to which the screen 43 is connected by computer cable or Wi-Fi system, where this information concerns the future schedule of the practitioner, its clients or the treatments to be administered for example, or it may be composed of urgent messages.

It is also possible to arrange that the screen 43 should be composed of a touch screen, in which case it can be used as a computer terminal and can then allow the circulation of information from the treatment cabin to the centralised computer system.

As an example, each treatment administered in the cabin, or each product chosen in the cabin by a client, can be identified by the practitioner on the screen 43, with the corresponding information being transmitted to the reception desk of the beauty salon, in order to allow automatic preparation of the bill for the treatment administered to this client and the products purchased by the latter.

1. An aesthetic treatment installation that includes a treatment table with at least one head and one median part; treatment appliances connected to an electrical source; accessories including power leads for equipping the treatment appliances; devices for the control and/or adjustment of the treatment appliances, where the treatment appliances are equipped with inputs for the control and/or adjustment signals and outputs presenting output signals for delivery to respective ones of the accessories, first storage resources associated with the treatment table, each said first storage resource accommodating at least one of said treatment appliances; second storage resources provided at a head of the treatment table and designed to accommodate the accessories; and a control panel placed at the head of the treatment table and on which are grouped the control and/or adjustment devices, said panel also being equipped with sockets for connection of the power leads; and connection resources positioned under the treatment table in order to connect the control and/or adjustment devices of the panel to the corresponding inputs of the appliances, and the sockets of the panel to the corresponding outputs of the appliances.

2. A treatment installation according to claim 1 wherein the first storage resources are composed of boxes or drawers.

3. A treatment installation according to claim 1, wherein the first storage resources are mounted under a lower face of a median part of the treatment table.

4. A treatment installation according to claim 1, wherein the second storage resources are composed of drawers or small tables.

5. A treatment installation according to claim 1, wherein the second storage resources are mounted under a lower face of the head of the treatment table.

6. A treatment installation according to claim 1, wherein the panel includes display devices that are capable of displaying an operational state of the appliances, and said display devices are connected to the appliances by the connection resources.

7. A treatment installation according to claim 1, further comprising a display screen placed at the head of the treatment table.

8. A treatment installation according to claim 7, wherein the display screen is built into the control panel.

9. A treatment installation according to claim 7, wherein said display screen is composed of a touch screen.

10. A treatment installation according to claim 7, further comprising a centralised computer system to which said display screen is connected.
